SEQUENCE LISTING (3 sequences)

SEQ ID NO: 1 - protein, 15 aa, Homo sapiens
CAPDEELLSCSSFSR

SEQ ID NO: 2 - protein, 13 aa, Homo sapiens
DVINEAWFPEDQR

SEQ ID NO: 3 - protein, 15 aa, Homo sapiens
ILHVFHGLLPGFLVK

(19) United States
(12) Patent Application Publication
(10) Pub. No.: US 2015/0080463 A1
(43) Pub. Date: Mar. 19, 2015

(54) METHODS FOR PREDICTING CARDIOVASCULAR EVENTS AND MONITORING TREATMENT USING PCSK9

(71) Applicant: Pieter Muntendam, Boxford, MA (US)

(72) Inventor: Pieter Muntendam, Boxford, MA (US)

(73) Assignee: BG MEDICINE, INC., Waltham, MA (US)

(21) Appl. No.: 13/634,616

(22) PCT Filed: Mar. 16, 2011
(86) PCT No.: PCT/US11/28721

§ 371 Date: Jan. 23, 2013

Related U.S. Application Data
(60) Provisional application No. 61/314,316, filed on Mar. 16, 2010.

(51) Int. Cl. G01N 33/573; G06F 19/00; A61K 31/351

(52) U.S. Cl. CPC G01N 33/573; A61K 31/351; G06F 19/3431; G01N 2800/52; G01N 2333/96433; G01N 2800/324
USPC 514/460; 435/24; 435/7.4; 435/6.12; 435/6.11; 506/9; 506/2; 702/19

(57) ABSTRACT

Methods are provided for diagnosing the risk of a cardiovascular event in a patient. In some embodiments, the method includes measuring the level of proprotein convertase subtilisin kexin type 9 (PCSK9) in a sample obtained from a patient and comparing the measured level of PCSK9 to a control. Also provided are methods of selecting a therapy for a patient prior to administration of the therapy. In some embodiments, the method includes measuring a PCSK9 blood concentration in a sample from a patient to determine the presence or absence of a PCSK9 blood concentration indicative of responsiveness to an inhibitor of 3-hydroxy-3-methylglutaryl-coenzyme A reductase. Further provided are methods for assessing the efficacy of a therapy being administered to a patient. In certain embodiments, the method includes detecting a change in a PCSK9 blood concentration in a sample from a patient, wherein a change in detected levels is indicative of whether the therapy is efficacious.

### REFERENCE TO RELATED APPLICATIONS

This application claims priority to and the benefit of U.S. Provisional Patent Application No. 61/314,316, filed on Mar. 16, 2010, the entire contents of which are hereby incorporated by reference herein.

### REFERENCE TO SEQUENCE LISTING

This application includes as part of the originally filed subject matter a Sequence Listing electronically submitted via EFS-Web as a single text file named “BYG-041PC_ST25.txt”. The Sequence Listing text file was created on Mar. 15, 2011 and is 1 kb in size. The contents of the Sequence Listing are hereby incorporated by reference.

### BACKGROUND

Atherosclerotic cardiovascular disease (CVD) and cardiovascular events (CVE) including, for example, myocardial infarction (MI), are predominantly caused by modifiable risk factors but nonetheless remains the leading cause of death and severe disability worldwide. (Yusuf et al., Effect of potentially modifiable risk factors associated with myocardial infarction in 52 countries (the INTERHEART study): case-control study, Lancet, 364:937-52 (2004)) To prevent the disease, contemporary American and European guidelines recommend an integrated two-step approach in which risk assessment (prediction) is followed by individualized risk reduction (therapy), if needed; the higher the risk, the more aggressive the prescribed preventive care. (Third Report of the National Cholesterol Education Program (NCEP) Expert Panel on Detection, Evaluation, and Treatment of High Blood Cholesterol in Adults (Adult Treatment Panel III) final report, Circulation, 106:3143-421 (2002); European guidelines on cardiovascular disease prevention in clinical practice: executive summary, Eur. Heart J., 28:2375-414 (2007)).

Risk assessment in primary prevention of atherosclerotic cardiovascular disease has not changed dramatically in the last 40 years. It remains based upon the risk factor concept introduced by the Framingham Heart Study in the 1960's. (Kannel et al., Factors of risk in the development of coronary heart disease—six year follow-up experience: The Framingham Study, Ann. Intern. Med., 55:33-50 (1961)). Because individual risk factors such as plasma cholesterol and blood pressure have low independent predictive ability (Ware. The limitations of risk factors as prognostic tools, N. Engl. J. Med., 355:2615-7 (2006)), they have been combined to generate global risk assessment measures such as the Framingham Risk Score (FRS) and the European SCORE (Systematic Coronary Risk Evaluation). (Third Report of the National Cholesterol Education Program (NCEP) Expert Panel on Detection, Evaluation, and Treatment of High Blood Cholesterol in Adults (Adult Treatment Panel III) final report, Circulation, 106:3143-421 (2002); European guidelines on cardiovascular disease prevention in clinical practice: executive summary, Eur. Heart J., 28:2375-414 (2007)).

In addition, the medical practice of administration of pharmacological agents to individuals with cardiovascular disease, such as hyperlipidemia, has revealed treatment responses that differ among different individuals, or at different times, or with different dosages. For example, HMG-CoA reductase inhibitors, or statins, are a class of hypolipidemic drugs that are prescribed to treat hyperlipidemia and reduce cardiovascular disease risk by lowering blood total cholesterol levels in humans by inhibiting the enzyme HMG-CoA reductase. Inhibition of this enzyme decreases cholesterol synthesis in the liver and increases low-density lipoprotein (LDL) receptors, resulting in an increased clearance of LDL from circulation. Individual response to statins may be assessed by measurement of blood concentrations of lipids and/or lipoproteins. It has been found that individual response to statins is highly variable. It has been postulated that genetic variation may modify both statin efficacy and susceptibility to statin-induced adverse drug reactions; however, definitive genetic variations associated with statin response have been elusive (Mangravite et al., Pharmacogenomics of statin response, Curr. Opin. Mol. Ther., 10(6):555-61 (2008)) Similar varying treatment responses characterize other pharmacological therapies for cardiovascular disease, such as peroxisome proliferator activated receptor alpha agonists, or fibrates.

A medically unsatisfactory pharmacological treatment effect for an individual with any pharmacological treatment for cardiovascluar disease and/or hyperlipidemia may result in a physician changing prescribed dosage and/or frequency, or attempting an alternate therapeutic strategy. Physicians routinely prescribe treatment regimens without knowledge of how an individual will respond to the treatment. As such, a trial and error treatment strategy is initiated, often at great cost and at the expense of severe side effects and loss of valuable treatment time.

Thus, there is a need to improve the prediction of cardiovascular events, such as MI. There is also a need to predict individual response to, and monitor individual response to, pharmacological therapies for hyperlipidemia and cardiovascular disease.

### SUMMARY

It has been discovered that proprotein convertase subtilisin kexin type 9 (PCSK9) levels can be used to evaluate a patient's expected response to drug treatment for cardiovascular disease (e.g., hyperlipidemia or high cholesterol). For example, a patient's PCSK9 levels can be measured prior to drug administration (or after a sufficient washout period) to determine whether the patient is likely to respond to the selected drug (e.g., a statin). In addition, levels of PCSK9 in a patient are associated with a higher risk of cardiovascular disease and/or cardiovascular events. Moreover, it has been discovered that PCSK9 levels increase in response to treatment with certain drugs or medications (e.g., statins), and thus a patient's PCSK9 level can be monitored to assess whether the patient is complying with a prescribed treatment regimen. PCSK9 levels also can increase over time in patients who respond favorably to treatment with certain drugs or medications (e.g., statins). Thus, a patient's PCSK9 levels can be monitored to assess whether a drug is efficacious.

In various embodiments, a method is provided for predicting response to treatment with a statin. The method can include measuring a proprotein convertase subtilisin kexin type 9 (PCSK9) level in a sample obtained from a patient prior to the patient taking a statin or after a sufficient washout period (e.g., about 6 weeks) has elapsed for the patient. A measured PCSK9 level falling within a predetermined target range or under a maximum threshold can be indicative that the patient is likely to respond favorably to treatment with a statin. On the other hand, a measured PCSK9 level above the predetermined target range (e.g., about 0 nanomoles per liter to about 7 nanomoles per liter) or a maximum threshold can be indicative that the patient is likely to have an attenuated response to treatment with a statin. In some embodiments, the measured PCSK9 level can be an absolute concentration or it can be a normalized concentration.

In various embodiments, a method is provided for assessing patient compliance with a treatment regimen. The method can include detecting a change over time in proprotein convertase subtilisin kexin type 9 (PCSK9) levels measured in samples obtained from a patient, the patient being prescribed a treatment regimen that includes a medication to treat cardiovascular disease. A change in PCSK9 levels over time can be indicative of whether the patient is following the treatment regimen.

In various embodiments, a method is provided for assessing patient compliance with a treatment regimen. The method can include measuring a first proprotein convertase subtilisin kexin type 9 (PCSK9) level in a sample obtained from a patient at a first time, the patient being prescribed a treatment regimen that includes a medication to treat cardiovascular disease; measuring a second PCSK9 level in a sample obtained from the patient at a second time; and comparing the first PCSK9 level to the second PCSK9 level. A change in PCSK9 levels over time can be indicative of whether the patient is following the treatment regimen.

In methods of the present teachings, the medication, treatment, therapy or drug can include an anti-hyperlipidemia and/or an anti-hypercholesterolemia pharmacological agent, for example, a statin. Non-limiting examples of statins include: atorvastatin, cerivastatin, fluvastatin, lovastatin, mevastatin, pitavastatin, pravastatin, rosuvastatin, and simvastatin.

Methods of the present teachings can further include determining whether the patient is likely to respond favorably to the medication by measuring PCSK9 levels in a sample obtained from the patient prior to the patient taking the medication, or after a sufficient washout period for the patient. A measured PCSK9 level falling within a predetermined target range can be indicative that the patient is likely to respond favorably to the medication.

In methods of the present teachings, the change in PCSK9 levels over time can be a decrease in PCSK9 levels or no change in PCSK9 levels, which change (or lack of change) can be indicative of the patient not being in compliance with the treatment regimen.

In methods of the present teachings, the change in PCSK9 levels over time can be an increase in PCSK9 levels, which change can be indicative of the patient being in compliance with the treatment regimen.

Methods of the present teachings can further include notifying at least one of the patient, a physician, healthcare provider, a medical insurance provider, and an insurance provider, that the patient is not in compliance with the treatment regimen.

Methods of the present teachings can further include transmitting, displaying, storing, or printing, or outputting to a user interface device, a computer readable storage medium, a local computer system or a remote computer system, information indicative of whether or not the patient is following the treatment regimen.

In various embodiments, a method is provided for assessing the efficacy of a proprotein convertase subtilisin kexin type 9 (PCSK9) modulating agent. The method can include detecting a change over time in PCSK9 levels measured in samples obtained from a patient, the patient being administered an agent that modulates PCSK9 levels to treat cardiovascular disease. A change in PCSK9 levels over time can be indicative of whether the PCSK9 modulating agent is efficacious.

In various embodiments, a method is provided for assessing the efficacy of a proprotein convertase subtilisin kexin type 9 (PCSK9) modulating agent. The method can include measuring a first PCSK9 level in a sample obtained from a patient at a first time, the patient being administered an agent that modulates PCSK9 levels to treat cardiovascular disease; measuring a second PCSK9 level in a sample obtained from the patient at a second time; and comparing the first PCSK9 level to the second PCSK9 level. A change in PCSK9 levels over time can be indicative of whether the PCSK9 modulating agent is efficacious.

In various embodiments, a computer system is provided for predicting patient response to treatment with a statin. The computer system can include an electronic memory device and an electronic processor in communication with the memory device. The memory device can include instructions that when executed by the processor cause the processor to: query a proprotein convertase subtilisin kexin type 9 (PCSK9) level measured in a sample obtained from a patient prior to the patient taking a statin or after a sufficient washout period has elapsed for the patient; compare the measured PCSK9 level to a threshold range; and generate a notification indicative of whether the measured PCSK9 level is within the target range. A measured PCSK9 level falling within a predetermined target range can be indicative that the patient is likely to respond favorably to treatment with a statin. A measured PCSK9 level above the predetermined range can be indicative that the patient is likely to have an attenuated response to treatment with a statin.

In various embodiments, a computer system is provided for monitoring patient compliance with a prescribed treatment regimen. The prescribed treatment regimen can include a medication to treat cardiovascular disease. The computer system can include an electronic memory device and an electronic processor in communication with the memory device. The memory device includes instructions that when executed by the processor cause the processor to: query a reference proprotein convertase subtilisin kexin type 9 (PCSK9) level; query a first PCSK9 level measured in a first sample obtained at a first time from a patient; compare the reference PCSK9 level to the first PCSK9 level; and generate a notification indicative of whether the reference PCSK9 level is lower than, higher than, or the same as the first PCSK9 level. The notification can be indicative of whether the patient is following the treatment regimen. In some embodiments, the reference PCSK9 level is a measurement of the PCSK9 level in a sample obtained from the patient prior to the first time.

In various embodiments, a computer system is provided for assessing the efficacy of a proprotein convertase subtilisin kexin type 9 (PCSK9) modulating agent. The computer system can include an electronic memory device and an electronic processor in communication with the memory device. The memory device includes instructions that when executed by the processor cause the processor to: query a reference PCSK9 level; query a first PCSK9 level measured in a first sample obtained at a first time from a patient; compare the reference PCSK9 level to the first PCSK9 level; and generate a notification indicative of whether the reference PCSK9 level is lower than, higher than, or the same as the first PCSK9 level. The notification can be indicative of whether the PCSK9 modulating agent is efficacious. In some embodiments, the reference PCSK9 level is a measurement of the PCSK9 level in a sample obtained from the patient prior to the first time.

In various embodiments, a method is provided for diagnosing the risk of a cardiovascular event in a patient. The method can include measuring the level of proprotein convertase subtilisin kexin type 9 (PCSK9) in a patient sample; comparing the measured level of PCSK9 to a control; and identifying, based on the comparison, an increased risk of a cardiovascular event in the patient if the measured level is less than the control, and a decreased risk of a cardiovascular event in the patient if the measured level is equal to or greater than the control.

In various embodiments, a method is provided for diagnosing the risk of a cardiovascular event in a patient. The method can include measuring the level of proprotein convertase subtilisin kexin type 9 (PCSK9) in a patient sample; comparing the measured level of PCSK9 to a control; and transmitting, displaying, storing, or printing; or outputting to a user interface device, a computer readable storage medium, a local computer system, or a remote computer system, information related to the risk of a cardiovascular event in the patient based on the comparison. A measured level less than the control can be indicative of an increased risk of a cardiovascular event in the patient, and a measured level greater than or equal to the control can be indicative of a decreased risk of a cardiovascular event in the patient. In some embodiments, the information can include at least one of the measured level, the control, the comparison, and equivalents thereof.

In various embodiments, a method is provided for diagnosing the risk of a cardiovascular event in a patient. The method can include comparing the measured level of proprotein convertase subtilisin kexin type 9 (PCSK9) in a patient sample to a control; transmitting, displaying, storing, or printing; or outputting to a user interface device, a computer readable storage medium, a local computer system, or a remote computer system, information relating to the risk of a cardiovascular event in the patient based on the comparison. A measured level less than the control can be indicative of an increased risk of a cardiovascular event in the patient, and a measured level greater than or equal to the control can be indicative of a decreased risk of a cardiovascular event in the patient. In some embodiments, the information can include at least one of the measured level, the control, the comparison, and equivalents thereof.

In various embodiments, a method of treatment is provided. The method can include determining, based on a comparison of the measured level of proprotein convertase subtilisin kexin type 9 (PCSK9) in a patient sample to a control, a risk of a cardiovascular event in the patient; and recommending, authorizing, or administering treatment if the patient is identified as having an increased risk of a cardiovascular event. A measured level less than the control can be indicative of an increased risk of a cardiovascular event in the patient, and a measured level greater than or equal to the control can be indicative of a decreased risk of a cardiovascular event in the patient.

In various embodiments, a method of treatment is provided. The method can include identifying a patient as having an increased or decreased risk of a cardiovascular event based on the measured level of proprotein convertase subtilisin kexin type 9 (PCSK9) in a patient sample; and recommending, authorizing, or administering treatment if the patient is identified as having an increased risk of a cardiovascular event.

In methods of treatment of the present teachings, the measured level of PCSK9 can be a measured level of PCSK9 RNA, a measured level of PCSK9 protein, or a combination thereof.

In methods of treatment of the present teachings, the measured level of PCSK9 can be a measured level of PCSK9 protein.

In methods of treatment of the present teachings, the measured level of PCSK9 can be determined by measuring one or more proteolytic peptides of PCSK9 by mass spectrometry.

In methods of treatment of the present teachings, the patient sample can include blood, serum, or plasma.

In methods of treatment of the present teachings, the patient can be a mammal, such as a human.

In methods of treatment of the present teachings, the cardiovascular event can include a myocardial infarction, stroke (including ischemic stroke and hemorrhagic stroke), heart failure, angina pectoris, venous thrombosis, arterial thrombosis, thromboembolism, cardiac arrest, cardiac thrombus, transient ischemic attack, development of cardiac valvular disease, development of peripheral artery disease, death, and combinations thereof.

In methods of treatment of the present teachings, the cardiovascular event can be a near-term myocardial infarction. In various embodiments, a near-term myocardial infarction event is a myocardial infarction event that occurs within about four years from the date the patient sample is taken.

In methods of treatment of the present teachings, the control can correspond to a PCSK9 level in one or more normal individuals.

In methods of treatment of the present teachings, the comparison can include a weighted analysis of the measured level of PCSK9 and one or more clinical risk factors for the patient. The one or more clinical risk factors can be selected from smoking status, diabetes mellitus, family history of premature myocardial infarction, body mass index, physical activity, nonfasting total cholesterol, HDL cholesterol, LDL cholesterol, and triglycerides.

In various embodiments, a method is provided for selecting a therapy for a human prior to administration of the therapy. The method can include measuring a proprotein convertase subtilisin kexin type 9 (PCSK9) blood concentration in a sample from the human, thereby to determine the presence or absence of a PCSK9 blood concentration indicative of responsiveness to an inhibitor of 3-hydroxy-3-methylglutaryl-coenzyme A reductase.

In methods of the present teachings, the sample can include blood, serum or plasma.

Methods of the present teachings can further include repeatedly administering the inhibitor to the patient.

In various embodiments, a method is provided for treating a human. The method can include repeatedly administering a 3-hydroxy-3-methylglutaryl-coenzyme A reductase inhibitor to a patient having a determined PCSK9 blood concentration indicative of a survival-enhancing response to the inhibitor.

Methods of the present teachings can further include monitoring the patient's PCSK9 blood concentration over the course of the therapy.

In methods of the present teachings, the inhibitor (e.g., a statin) is administered in an amount sufficient to inhibit progression or development of cardiovascular disease or a cardiovascular event. For example, the inhibitor can be administered in a survival-enhancing amount. Non-limiting examples of statins include one or more of atorvastatin, cerivastatin, fluvastatin, lovastatin, mevastatin, pitavastatin, pravastatin, rosuvastatin, and simvastatin. For example, rosuvastatin can be administered at a dose of between 5 and 40 mg/day, and atorvastatin can be administered at a dose of between 10 and 80 mg/day.

In methods of the present teachings, the patient can have a PCSK9 blood concentration determined to be within a target range (e.g., about 0 nanomoles per liter to about 7 nanomoles per liter). In some embodiments, the patient can have a PCSK9 blood concentration determined to be above a minimum threshold. The minimum threshold can be, for example, more than 1 nanomole per liter, between 1 and 1.5 nanomoles per liter, between 1.5 and 2 nanomoles per liter, between 2 and 2.5 nanomoles per liter, between 2.5 and 3 nanomoles per liter, and/or more than 3 nanomoles per liter. In some embodiments, the patient can have a PCSK9 blood concentration determined to be below a maximum threshold. The maximum threshold can be, for example, below 7 nanomoles per liter, below 6 nanomole per liter, below 4 nanomoles per liter, between 3 and 4 nanomoles per liter, between 2.5 and 3 nanomoles per liter, between 2 and 2.5 nanomoles per liter, and/or between 1.5 and 2 nanomoles per liter.

### BRIEF DESCRIPTION OF DRAWING

The present teachings described herein will be more fully understood from the following description of various illustrative embodiments, when read together with the accompanying drawings. It should be understood that the drawings described below are for illustration purposes only and are not intended to limit the scope of the present teachings in any way.

FIG. 1 is a schematic of a colorimetric assay for determining PCSK9 concentration, in accordance with an illustrative embodiment.

FIG. 2 is a graph showing percent change in blood serum levels of PCSK9 between week 0 and week 12, in six treatment groups and one control, in accordance with an illustrative embodiment.

FIG. 3 is box plot comparing measured plasma concentration of PCSK9 in healthy individuals (control) and affected individuals (case), in accordance with an illustrative embodiment.

### DETAILED DESCRIPTION

It has been discovered that levels of proprotein convertase subtilisin kexin type 9 (PCSK9) in a patient are associated with a higher risk of cardiovascular disease and/or cardiovascular events. More specifically, PCSK9 levels can be informative of and indicate the extent to which a patient is at risk of suffering a cardiovascular event (e.g., a myocardial infarction (MI)) in the future. For example, in certain embodiments, reduced measured PCSK9 levels as compared to a normal control indicate that the patient is at an increased risk of having a cardiovascular event, such as MI, in the future. As a result, the patient can modify his or her behavior and/or seek medical intervention to reduce the likelihood of the cardiovascular event actually occurring.

It also has been discovered that PCSK9 levels can be used to evaluate a patient's expected response to drug treatment for cardiovascular disease (e.g., hyperlipidemia or high cholesterol). For example, a patient's PCSK9 levels can be measured prior to drug administration (or after a sufficient washout period) to determine whether the patient is likely to respond to the selected drug (e.g., a statin). A patient whose PCSK9 levels fall within a target range has an increased likelihood of responding favorably to the selected treatment. Thus, physicians and patients can use this information to make informed decisions before selecting a treatment regimen.

It further has been discovered that PCSK9 levels increase in response to treatment with certain drugs or medications (e.g., statins) and, thus, a patient's PCSK9 level can be monitored to assess whether the patient is complying with a prescribed treatment regimen. For example, an increase in PCSK9 levels over time, or as compared to a baseline level or a control, is indicative that the patient is complying with the treatment regimen. On the other hand, no change and/or a decrease in PCSK9 levels is indicative that the patient is not complying with the treatment regimen, which might be caused by the patient skipping doses, taking too little medication, or taking medication at wrong and/or inconsistent times. Accordingly, physicians, patients, and insurers can keep apprised of whether treatment regimens are being followed properly.

In addition, PCSK9 levels can increase over time in patients who respond favorably to treatment with certain drugs or medications (e.g., statins). Thus, a patient's PCSK9 levels can be monitored to assess whether a drug is efficacious. For example, an increase in PCSK9 levels over time, or as compared to a baseline level or control, is indicative that the drug is efficacious. On the other hand, no change and/or a decrease in PCSK9 levels is indicative that the drug is not efficacious. Accordingly, physicians and patients can make informed decisions about whether to continue and/or modify treatment with the drug, or to switch to another treatment that may be more efficacious.

In various embodiments, PCSK9 levels in a patient are used as a biomarker. In general, a “biomarker” can be any biological feature or variable whose qualitative or quantitative presence, absence, or level in a biological system such as a human is an indicator of a biological state of the system. Accordingly, biomarkers can be useful to assess the health state or status of an individual by comparing the measured level of one or more biomarkers in a patient or a patient sample to a control. In addition, multiple biomarker levels can be analyzed using a weighted analysis or algorithm to generate a risk score for an individual. The risk score can be indicative of the likelihood that the individual will suffer a cardiovascular event (e.g., a MI).

As described in detail in the Examples, the present teachings provide methods for predicting whether a patient will respond favorably to a drug or medication prior to administering the drug or medication. This information can allow physicians to more quickly select appropriate treatments, which potentially can save lives and also can reduce resources spent on ineffective or not optimally effective treatments. Moreover, methods are provided for monitoring patient compliance with prescribed treatment regimens, which can permit the patient, a physician, or an insurer to take corrective action. The present teachings also can be used to identify individuals who appear healthy but may be at risk for experiencing a cardiovascular event, such as MI. Armed with this information, individuals at risk can take proactive steps such as exercising, dieting, and/or seeking medical intervention to reduce the likelihood of suffering that cardiovascular event in the future. Thus, the present teachings can be used more accurately to predict cardiovascular events and possibly save lives.

It should be understood that the present teachings are not limited to myocardial infarction, but may be applicable to cardiovascular and atherothrombotic events generally. Atherothrombotic events include, but are not limited to, MI, stroke, visceral or limb infarction, and combinations thereof. Atherothrombotic events may occur, for example, in subjects who are asymptomiatic, or in subjects who have been diagnosed with a disease, for example, coronary artery disease, cerebrovascular disease, and/or peripheral arterial disease. Cardiovascular events include, but are not limited to, stroke (including ischemic stroke and hemorrhagic stroke), heart failure, angina pectoris, venous thrombosis, arterial thrombosis, thromboembolism, cardiac arrest, cardiac thrombus, transient ischemic attack, development of cardiac valvular disease, development of peripheral artery disease, death, and combinations thereof.

As described above, the term “biomarker” refers to any biological feature or variable whose qualitative or quantitative presence, absence, or level in a biological system such as a human is an indicator of a biological state of the system. For example, a biomarker of an organism can be useful, alone or in combination with other biomarkers and/or clinical risk factors, to measure the initiation, progression, severity, pathology, aggressiveness, grade, activity, disability, mortality, morbidity, disease sub-classification or other underlying feature of one or more biological processes, pathogenic processes, diseases, or responses to therapeutic intervention. Virtually any biological compound that is present in a sample and that can be isolated from, or measured in, the sample can be used as a biomarker. Non-limiting examples of classes of biomarkers include a polypeptide, a proteoglycan, a glycoprotein, a lipoprotein, a carbohydrate, a lipid, a nucleic acid, an organic on inorganic chemical, a natural polymer, a metabolite, and a small molecule. A biomarker also can include a physical measurement of the human body, such as blood pressure and cell counts, as well as the ratio or proportion of two or more biological features or variables.

The “level” or “amount” of a biomarker can be determined by any method known in the art and will depend in part on the nature of the biomarker. It should be understood that the amount of the biomarker need not be determined in absolute terms, but can be determined in relative terms. Thus, in some embodiments, (measured) levels can be absolute concentrations measured in a sample obtained from a patient, and in some embodiments, (measured) levels can be normalized concentrations measured in a sample obtained from a patient. In addition, the amount of the biomarker can be expressed by its concentration in a biological sample, for example, a sample obtained from a mammal such as a human, by the concentration of an antibody that binds to the biomarker, or by the functional activity (i.e., binding or enzymatic activity) of the biomarker.

The term “near-term” means within about zero to about six years from a baseline, where the baseline is defined as the date on which a patient sample is taken for analysis. For example, near-term includes within about one week, about one month, about two months, about three months, about six months, about nine months, about one year, about two years, about three years, about four years, about five years, or about six years from a baseline.

The term “near-term risk” means the risk that a patient will experience a cardiovascular event within the near-term.

The terms “reference,” “control” and “standard” can refer to an amount of a biomarker in a healthy individual or a control population or to a risk score derived from one or more biomarkers in a healthy individual or a control population. The amount of a biomarker can be determined in a sample of a healthy individual, or can be determined in samples of a control population. A control population can be a group of healthy individuals, individuals lacking diagnosis of the particular disease for which the biomarker is indicative, and/or a sub-population of such individuals where the sub-population is selected based on the background of the patient, for example, based on gender, age, ethnicity, or other distinguishing or clinically relevant features.

The term “sample” refers to any biological sample from an individual (e.g., a patient), including body fluids, blood, blood plasma, blood serum, sebum, cerebrospinal fluid, bile acid, saliva, synovial fluid, pleural fluid, pericardial fluid, peritoneal fluid, sweat, feces, nasal fluid, ocular fluid, intracellular fluid, intercellular fluid, lymph urine, tissue, liver cells, epithelial cells, endothelial cells, kidney cells, prostate cells, blood cells, lung cells, brain cells, adipose cells, tumor cells, and mammary cells. The sources of biological sample types can be different subjects; the same subject at different times; the same subject in different states, e.g., prior to drug treatment and after drug treatment; different sexes; different species, for example, a human and a non-human mammal; and various other permutations. Further, a biological sample type can be treated differently prior to evaluation such as using different work-up protocols.

The present teachings provide, in part, a method of diagnosing the risk of a cardiovascular event (e.g., MI or near-term MI) in an individual such as a human patient. In various embodiments, the method generally includes measuring the level (or using a measured level) of PCSK9 in a patient sample (e.g. a sample obtained from a patient) and comparing the measured level to a control. Based on the comparison, the patient can be identified as being at an increased risk of having a cardiovascular event if the patient's PCSK9 level is lower than the control. Conversely, the patient is at a decreased risk of having a cardiovascular event if the patient's PCSK9 level is equal to or greater than the control. In some embodiments, the method includes transmitting, displaying, storing, or printing—or outputting to a user interface device, a computer readable storage medium, a local computer system, or a remote computer system—information related to the risk of a cardiovascular event in the patient.

In some embodiments, the method can include a weighted analysis of PCSK9 levels together with one or more clinical risk factors. Clinical risk factors include, but are not limited to, smoking status, diabetes mellitus, family history of premature myocardial infarction, body mass index, physical activity, nonfasting total cholesterol, HDL cholesterol, LDL cholesterol, and triglycerides. It also will be appreciated that one or more biomarker levels can be measured in addition to PCSK9 levels. Multimarker analyses are known and can improve the accuracy of diagnosis and monitoring. Such biomarkers include, for example, low-density lipoprotein cholesterol, high-density lipoprotein cholesterol, total cholesterol, very low-density lipoprotein cholesterol, non-high-density lipoprotein cholesterol, non-low-density lipoprotein cholesterol, non-very low-density lipoprotein cholesterol, triglycerides, low-density lipoprotein, high-density lipoprotein, very low-density lipoprotein, soluble low-density lipoprotein receptor, cholesteryl ester transfer protein, apolipoprotein A (including the subclasses of apoliprotein A-I, apoliprotein A-II, apoliprotein A-IV, and apoliprotein A-V), apoliprotein B (including the subclasses of apoliprotein B48 and apoliprotein B100), apoliprotein C (including the subclasses of apoliprotein C-I, apoliprotein C-II, apoliprotein C-III, and apoliprotein C-IV), apoliprotein E, apoliprotein D, apoliprotein H.

Establishing a reference risk score or a “cutoff” score for weighted analysis of one or more biomarkers and/or clinical risk factors is known in the art. (Szklo et al., Epidemiology: beyond the basics (Second Ed.; Sudbury, Mass.: Jones and Bartlett Publishers (2007)); Schlesselman, Case-Control Studies (New York: Oxford University Press (1982)); Anderson et al., Cardiovascular disease risk profiles, Am. Heart J., 121:293-8 (1991); Eichler et al., Prediction of first coronary events with the Framingham score: a systematic review. Am. Heart J., 153(5):722-31, 731.e1-8 (2007); Hoffmann et al., Defining normal distributions of coronary artery calcium in women and men from the Framingham Heart Study, Am. J. Cardiol., 102(9):1136-41, 1141.e1. (2008))

The present teachings also provide methods for treating patients. In some embodiments, the method includes determining, based on a comparison of the measured level of PCSK9 in a patient sample to a control, a risk of a cardiovascular event in the patient; and recommending, authorizing, or administering treatment if the patient is identified as having an increased risk of a cardiovascular event. In various embodiments, the method can include identifying a patient as having an increased or decreased risk of a cardiovascular event based on the measured level of proprotein convertase subtilisin kexin type 9 (PCSK9) in a patient sample; and recommending, authorizing, or administering treatment if the patient is identified as having an increased risk of a cardiovascular event.

The present teachings also provide methods of determining whether a particular therapy for cardiovascular disease (e.g., statin treatment for high cholesterol) is suitable for a patient prior to administration of the therapy or after a sufficient washout period (e.g., 6-8 weeks without the therapy). In some embodiments, the method includes measuring a PCSK9 blood concentration in a sample from the patient to determine the presence or absence of a PCSK9 blood concentration indicative of responsiveness to a particular therapy. If the patient's PCSK9 level indicates that the patient may respond favorably to a particular therapy, then that therapy can be prescribed or administered with higher confidence. If the patient's PCSK9 level indicates that the patient may not respond or may respond unfavorably to the therapy, then an alternative therapy can be prescribed or administered.

Patients who have a PCSK9 blood concentration within a target range are considered to have a higher likelihood of responding favorably to a therapy. The target range can be, for example, between about 0 nanomole per liter (nM) to about 7 nM. In various embodiments, the target range has a minimum threshold of more than about 0 nM, more than about 0.5 nM, more than about 1 nM, between about 1-1.5 nM, between about 1-2 nM, between about 2 nM to about 2.5 nM, between about 2.5 nM to about 3 nM, or about 3 nM. In various embodiments, the target range has a maximum threshold of less than about 7 nM, about 6 nM, about 5 nM, about 4 nM, between about 3 nM to about 4 nM, between about 2.5 nM to about 3 nM, between about 2 nM to about 2.5 nM, between about 1.5 nM to about 2 nM or about 1.5 nM.

Measured PCSK9 levels can be absolute PCSK9 concentrations measured in a sample obtained from a patient, or measured PCSK9 levels can be normalized PCSK9 concentrations measured in a sample obtained from a patient.

In various embodiments, the measured levels of PCSK9 can be indicative of whether a patient will respond to a therapy, such as an anti-hypercholesterolemia and/or anti-hyperlipidemia medication. In some embodiments, the therapy includes a statin. Suitable statins include, by non-limiting example, atorvastatin, cerivastatin, fluvastatin, lovastatin, mevastatin, pitavastatin, pravastatin, rosuvastatin, and simvastatin. In certain embodiments, the statin is rosuvastatin, which can be administered at a dose between about 5 mg/day and about 40 mg/day. In some embodiments, the statin is atorvastatin, which can be administered at a dose of between about 10 mg/day and about 80 mg/day.

After it is determined that a patient is likely to respond favorably to therapy, the therapy (e.g., a drug) can be administered in an amount sufficient to inhibit progression or development of cardiovascular disease and/or a cardiovascular event and, preferably, in a survival-enhancing amount. In addition, the dosage and/or frequency of administration can be increased or decreased based on the patient's PCSK9 levels.

PCSK9 levels can be monitored before, during, or after administration of one or more doses of a therapy (e.g., a drug). For example, the therapy can be administered to the patient repeatedly, and PCSK9 levels measured after one or more of the administrations. In addition, PCSK9 levels can be monitored over the course of therapy, in case the patient later becomes non-responsive or responds unfavorably to the therapy. For example, biomarker levels can be monitored over time, such as in samples obtained from the patient at hourly, daily, weekly, biweekly, triweekly, monthly, bimonthly, trimonthly, semi-annual, annual, other, or variable intervals. The treatment regimen can be altered or discontinued if PCSK9 levels indicate that the patient is non-responsive or is responding unfavorably.

The present teachings also provide, in part, a kit useful for diagnosing the risk of a cardiovascular event in a patient (e.g., MI). Such a kit can contain, for example, one or more control or reference standards providing: baseline levels of PCSK9 or other selected biomarkers in a normal individual; baseline levels of PCSK9 in an individual who is known to be at risk for one or more cardiovascular events; baseline levels of PCSK9 in an individual who is responsive to a 3-hydroxy-3-methylglutaryl-coenzyme A reductase inhibitor; and baseline levels of PCSK9 in an individual who is non-responsive or responds unfavorably to a 3-hydroxy-3-methylglutaryl-coenzyme A reductase inhibitor.

The present teachings permit not only the diagnosis of the risk of a cardiovascular event, but also can be adapted to other uses. For example, biomarker levels and/or risk scores can be used to screen candidate drugs that mitigate the causative factors which lead to cardiovascular events. In this instance, treatment with candidate drugs can be monitored using one or more biomarker levels and/or a risk score.

Moreover, with any drug that already has been found effective to reduce the likelihood of future cardiovascular events, certain individuals may be responders and some may be non-responders. Accordingly, an individual's biomarker levels and/or risk score can be evaluated during treatment to determine if the drug is effective. For example, if the individual's risk score decreases in response to treatment, the individual may be responding to the treatment and therefore also may be at a decreased risk for experiencing a future cardiovascular event. Of course, there may not be any existing, known population of responders and non-responders so that the efficacy of drug treatment relating to future cardiovascular events can be monitored over time. To the extent the drug is not efficacious, its use can be discontinued and another drug and/or another therapy can be used in its place.

After a risk or likelihood of a future cardiovascular event is determined for a patient, information about the risk and/or a likelihood of a future cardiovascular event can be displayed or outputted to a user interface device, a computer readable storage medium, or a local or remote computer system. Such information can include, for example, a measured level of one or more biomarkers; a reference or control, for example, a control level, amount, and/or (risk) score; a comparison, for example, a comparison of the measured level to a control; a risk score; a likelihood of a cardiovascular event; or equivalents thereof (e.g., a graph, a figure, a symbol, etc.). Displaying or outputting information means that the information can be communicated to a user using any medium, for example, orally, in writing, by visual display and/or non-transitory computer readable medium, computer system, or other electronic device (e.g., smart phone, personal digital assistant (PDA), laptop, etc.). It will be clear to one skilled in the art that outputting information is not limited to outputting to a user or a linked external component(s), such as a computer system or computer memory, but can alternatively or additionally be outputted to internal components, such as any computer readable medium. Computer readable media can include, but are not limited to hard drives, floppy disks, CD-ROMs, DVDs, and DATs. Computer readable media does not include carrier waves or other wave forms for data transmission. It will be clear to one skilled in the art that the various sample evaluation and diagnosis methods disclosed and claimed herein, can, but need not be, computer-implemented, and that, for example, the displaying or outputting step can be done by, for example, communicating to a person orally or in writing (e.g., in handwriting).

According to various embodiments, a risk score, a likelihood of a cardiovascular event (e.g., MI), a measured biomarker level, a reference risk score, and/or equivalents thereof can be displayed on a screen or a tangible medium and/or can be transmitted to a person in a medical industry, a medical insurance provider, a health care provider, or to a physician.

In addition, any of the methods disclosed herein can be embodied as or into systems, for example, computer systems. A computer system can include an electronic memory device (e.g., a computer readable medium) and an electronic processor in communication with the memory device. The electronic memory device includes instructions for carrying out the disclosed methods. Such systems would be beneficial to healthcare providers and insurers, both for efficiently managing and tracking patient data and for managing patients.

In various embodiments, PCSK9 levels are measured. Many methods for detecting expression levels of a protein of interest, with or without quantitation, are well known and can be used with the present teachings. Examples of such assays are described below and can include, for example, immunoassays, chromatographic methods, and mass spectroscopy. Such assays can be performed on any biological sample including, among others, blood, plasma, and serum. In addition, many methods for detecting expression levels of a gene transcript (e.g., mRNA) of interest, with or without quantitation, are will known and can be used with the present teachings.

Biomarkers can be detected or quantified in a sample with the help of one or more “separation” analytical methods. For example, suitable separation methods can include a mass spectrometry method, such as electrospray ionization mass spectrometry (ESI-MS), ESI-MS/MS, ESI-MS/(MS)n (n is an integer greater than zero), matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)n, or atmospheric pressure photoionization mass spectrometry (APPI-MS), APPI-MS/MS, and APPI-(MS)n. Other mass spectrometry methods can include, for example, quadrupole, fourier transform mass spectrometry (FTMS) and ion trap. Spectrometric techniques that can also be used include resonance spectroscopy and optical spectroscopy.

Other suitable separation methods include chemical extraction partitioning, column chromatography, ion exchange chromatography, hydrophobic (reverse phase) liquid chromatography, isoelectric focusing, one-dimensional polyacrylamide gel electrophoresis (PAGE), two-dimensional polyacrylamide gel electrophoresis (2D-PAGE), or other chromatographic techniques, such as thin-layer, gas or liquid chromatography, or any combination thereof. In some embodiments, the biological sample to be assayed can be fractionated prior to application of the separation analytical technique.

Biomarkers can be detected or quantified by methods that do not require physical separation of the biomarkers themselves. For example, nuclear magnetic resonance (NMR) spectroscopy can be used to resolve a profile of a biomarker from a complex mixture of molecules. An analogous use of NMR to classify tumors is disclosed in Hagberg, NMR Biomed., 11:148-56 (1998), for example.

A biomarker in a sample also can be detected or quantified by combining the biomarker with a binding moiety capable of specifically binding the biomarker. The binding moiety can include a member of a ligand-receptor pair, i.e., a pair of molecules capable of having a specific binding interaction. The binding moiety also can include a member of a specific binding pair, such as antibody-antigen, enzyme-substrate, nucleic acid-nucleic acid, protein-nucleic acid, protein-protein, or other specific binding pairs known in the art. Binding proteins can be designed that have enhanced affinity for a target. Optionally, the binding moiety can be linked with a detectable label, such as an enzymatic, fluorescent, radioactive, phosphorescent or colored particle label. The labeled complex can be detected, e.g., visually or with the aid of a spectrophotometer or other detector, and/or can be quantified.

For example, PCSK9 can be detected using an immunoassay, such as an enzyme-linked immunosorbant assay (ELISA). An immunoassay can be performed by contacting a sample from a subject to be tested with an appropriate antibody under conditions such that immunospecific binding can occur if the biomarker is present. Subsequently, detecting and/or measuring the amount of any immunospecific binding by the antibody to the biomarker can be done. Other suitable immunoassays can be used, including, without limitation, competitive and non-competitive assay systems using techniques such as Western blots, radioimmunoassays, “sandwich” immunoassays, immunoprecipitation assays, immunodiffusion assays, agglutination assays, complement-fixation assays, immunoradiometric assays, and fluorescent immunoassays.

In a sandwich immunoassay, two antibodies capable of binding a biomarker generally are used, e.g., one immobilized onto a solid support, and one free in solution and labeled with a detectable chemical compound. Examples of chemical labels that can be used for the second antibody include radioisotopes, fluorescent compounds, and enzymes or other molecules that generate colored or electrochemically active products when exposed to a reactant or enzyme substrate. When a sample containing the biomarker is placed in this assaying system, the biomarker can bind to both the immobilized antibody and the labeled antibody, to form a “sandwich” complex on the support's surface. The complexed biomarker can be detected by washing away non-bound sample components and excess labeled antibody, and measuring the amount of labeled antibody complexed to the biomarker on the support's surface. Alternatively, the antibody which is free in solution can be labeled with a chemical moiety, for example, a hapten, which can be detected by a third antibody labeled with a detectable moiety that binds the free antibody or, for example, the hapten coupled thereto.

Both the sandwich immunoassay and tissue immunohistochemical procedures can be highly specific and very sensitive, provided that labels with good limits of detection are used. A detailed review of immunological assay design, theory and protocols can be found in numerous texts in the art, including Butt, Practical Immunology (ed. Marcel Dekker, New York (1984)) and Harlow et al. Antibodies, A Laboratory Approach (ed. Cold Spring Harbor Laboratory (1988)).

In general, immunoassay design considerations include preparation of antibodies (e.g., monoclonal or polyclonal antibodies) having sufficiently high binding specificity for the target to form a complex that can be distinguished reliably from products of nonspecific interactions. As used herein, the term “antibody” is understood to mean binding proteins, for example, antibodies or other proteins comprising an immunoglobulin variable region-like binding domain, having the appropriate binding affinities and specificities for the target. The higher the antibody binding specificity, the lower the target concentration that can be detected. As used herein, the terms “specific binding” or “binding specifically” are understood to mean that the binding moiety, for example, a binding protein, has a binding affinity for the target of greater than about 10⁵ M⁻¹, and preferably greater than about 10⁷M⁻¹.

In the present teachings, when an immunoassay is used to determine the level of PCSK9 present in a sample from a patient, the antibodies used in the assay can include, for example, H1 H316P, H1 M300N, H1 H313, H1 H314, H1 H315, H1 H317, H1 H318, H1 H320, H1 H321, H1 H334 (as described in International Application No. PCT/US2009/068013); KS-2C10 (Circulex Human PCSK9 ELISA Kit, Cat# CY-8079; Nagano, Japan); AX1, AX213, AX214; 3BX5C01, 3CX2A06, 3CX3D02, 3CX4B08 (as described in International Application No. PCT/US2007/023213); EB06682 (Everest Biotech, Oxfordshire, United Kingdon); ab28770, ab31762, ab52754, ab52755, ab95478, ab92753, ab42086, ab42085 (Abcam, Cambridge, Mass.); antibodies as described in Alborn et al., “Serum proprotein convertase subtilisin kexin type 9 is correlated directly with serum LDL cholesterol,” 53(10):1814-9 (2007). Moreover, one skilled in the art knows how to make or raise antibodies to a known target.

Target gene transcripts can be detected using numerous techniques that are well known in the art. Some useful nucleic acid detection systems involve preparing a purified nucleic acid fraction of a sample (e.g., a tumor biopsy, a cancer cell culture, a cell engrafted biocompatible matrix) and subjecting the sample to a direct detection assay or an amplification process followed by a detection assay. Amplification can be achieved, for example, by polymerase chain reaction (PCR), reverse transcriptase (RT), and coupled RT-PCR. Detection of a nucleic acid can be accomplished, for example, by probing the purified nucleic acid fraction with a probe that hybridizes to the nucleic acid of interest and in many instances, detection involves an amplification as well. Northern blots, dot blots, microarrays, quantitative PCR, quantitative RT-PCR, and real-time PCR are all well known methods for detecting a nucleic acid in a sample. Nucleic acids also can be amplified by ligase chain reaction, strand displacement amplification, self-sustained sequence replication or nucleic acid sequence-based amplification. Nucleic acids also can be detected by sequencing. The sequencing can use a primer specific to the target nucleic acid or a primer to an adaptor sequence attached to the target nucleic acid. Sequencing of randomly selected mRNA or cDNA sequences can provide an indication of the relative expression of a biomarker as indicated by the percentage of all sequenced transcripts containing the nucleic acid sequence corresponding to the biomarker.

Alternatively, a nucleic acid can be detected in situ, such as by hybridization, without extraction or purification. Gene transcripts can be detected on a medium-throughput basis, such as by using a qRT-PCR array (e.g., RT2 Endothelial Cell Biology PCR Array; SABiosciences, Baltimore, Md.). In addition, target gene transcripts can be detected on a high-thoughput basis using a number of well known methods, such as cDNA microarrays (Affymetrix, Santa Clara, Calif.), SAGE (Invitrogen, Carlsbad, Calif.), and high-throughput mRNA sequencing (Illumina Inc., San Diego, Calif.).

The present teachings are further illustrated by the following examples, which are provided for illustration and not limitation.

### Example 1

### Experimental Design

Concentrations of PCSK9 protein were measured in blood serum specimens of human subjects diagnosed with primary hypercholesterolemia or mixed hyperlipidemia who had participated in a multicenter, randomized, double-blind study to evaluate the lipid-altering efficacy and safety of pharmacological agents. Briefly, following a 6 to 8 week washout period, subjects were randomized to seven treatment arms: (i) 10 milligrams (mg) simvastatin administered daily for 4 weeks, followed by 20 mg simvastatin administered daily for 8 weeks; (ii) 20 mg simvastatin administered daily for 4 weeks, followed by 40 mg simvastatin administered daily for 8 weeks; (iii) 40 mg simvastatin administered daily for 12 weeks; (iv) 10 mg simvastatin co-administered with 1 g niacin and 20 mg laropiprant daily for 4 weeks, followed by 20 mg simvastatin co-administered with 2 g niacin and 40 mg laropiprant daily for 8 weeks; (v) 20 mg simvastatin co-administered with 1 g niacin and 20 mg laropiprant daily for 4 weeks, followed by 40 mg simvastatin co-administered with 2 g niacin and 40 mg laropiprant daily for 8 weeks; (vi) 40 mg simvastatin co-administered with 1 g niacin and 20 mg laropiprant daily for 4 weeks, followed by 40 mg simvastatin co-administered with 2 g niacin and 40 mg laropiprant daily for 8 weeks, and (vii) 1 g niacin and 20 mg laropiprant daily for 4 weeks, followed by 2 g niacin and 40 mg laropiprant daily for 8 weeks (with no simvastatin administration). Simvastatin, as all statins as a drug class, acts by inhibiting 3-hydroxy-3-methyl-glutaryl-CoA reductase, thereby modulating a biochemical pathway involved in the hepatic synthesis of cholesterol.

Levels of PCSK9 were assessed in blood serum specimens acquired just prior the start of the therapeutic administration (referred to as ‘Week 0’) and at 12 weeks after the end of the therapeutic administration period (referred to as ‘Week 12’). Approximately 50 to 75 subjects assigned to each of the seven treatment arms in the study provided blood samples.

Blood serum PCSK9 concentration was determined using a colorimetric assay. The PCSK9 concentrated calibrator (7.5 mg/mL), and the assay antibodies AX213 and AX1-Biotin were the reagents for the assay.

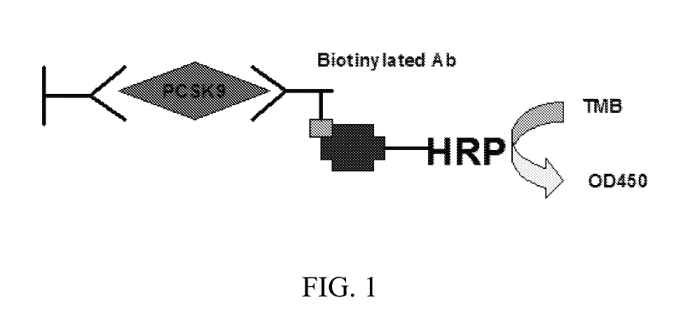

A concentration of 7.5 mg/mL assigned to the PCSK9 concentrated calibrator was used for calculating the needed dilution factors for preparing the calibrators used in the PCSK9 assay. A conversion factor of 1/78 was used to convert ng/mL to nM assuming a molecular weight for PCSK9 of 78,000 Daltons. FIG. 1 shows the colorimetric based detection system using horseradish peroxidase (HRP) enzyme chemistry for measuring PCSK9 in clinical samples. It takes advantage of the antibody pair as well as the binding reaction conditions. After adding the tetramethylbenzidine (TMB) substrate, the color development is measured by optical absorbance at a wavelength of 450 nm. Color development can be stopped by adding 100 microliters of 0.5M sulfuric acid.

Assay plates were prepared each day by adding 60 microliters of coating solution per well and incubated overnight at 4° C. Coating solution included 8.4 micrograms/mL of the capture antibody, AX213, in PBS buffer. The next morning, prior to use, they were blocked with 150 microliters of blocking solution (TBS buffer with 3% BSA and 0.05% Tweeen-20) per well for 1 hour at room temperature. Samples were diluted 100-fold in the assay diluent and measured in duplicate. Diluted samples, diluted controls, and calibrators were added into corresponding wells in the microtiter plate (50 μL per well) and incubated in the Boekel Jitterbug shaker (Boekel Scientific, Feasterville, Pa.) with shaking for 1 hour at 37° C. After washing with the wash buffer (400 μL per cycle for 4 cycles), 50 microliters of the biotinlyated detection antibody, AX1-Biotin, was added to each well at a final concentration of 1.0 μg/mL. The plate was incubated for 1 hour with shaking at room temperature followed by washing. 100 μL of Streptavidin-HRP conjugate was added into each well (10 ng/mL). The plate was incubated for 30 minutes without shaking at room temperature and was subsequently washed for 4 cycles with 400 μL of the wash buffer. 100 μL of TMB substrate was added into each well. The plate was incubated without shaking at room temperature for 20 minutes. After adding 100 μL of the stop solution, the plate was read at 450 nm.

### Example 2

### PCSK9 Levels Increase in Response to Treatment with a Statin

Samples and data were collected as described in Example 1.

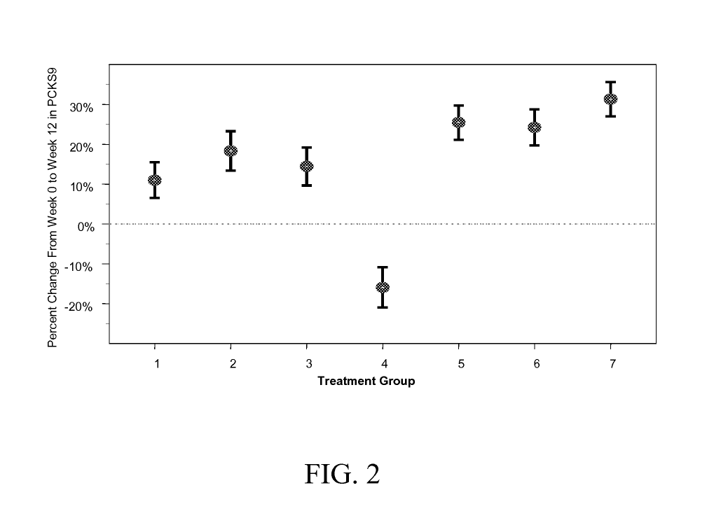

As shown in FIG. 2, it was observed that the levels of PCSK9 in blood serum increased between Week 0 (just prior the start of the therapeutic administration) and Week 12 (at the end of the therapeutic administration period) in the six groups receiving a therapy that included a statin, but decreased in the one group that did not receive a therapy that included a statin (Treatment Group ‘4’ in FIG. 2). FIG. 2 shows results of an analysis of covariance (ANCOVA) of the change in PCSK9 levels between Week 0 and Week 12, with the ANCOVA model adjusted for the covariates of treatment, gender, age (treated as a continuous variable), geographic region of subject's residency (United States or non-United States), and Week 0 level of PCSK9. Each circle indicates the calculated least squares mean for a given group, and the corresponding bars indicate the calculated standard error. The Treatment Group designations are as follows: (5) 10 milligrams (mg) simvastatin administered daily for 4 weeks, followed by 20 mg simvastatin administered daily for 8 weeks (N=74 subjects); (6) 20 mg simvastatin administered daily for 4 weeks, followed by 40 mg simvastatin administered daily for 8 weeks (N=67); (7) 40 mg simvastatin administered daily for 12 weeks (N=71); (1) 10 mg simvastatin co-administered with niacin and laropiprant daily for 4 weeks, followed by 20 mg simvastatin co-administered with niacin and laropiprant daily for 8 weeks (N=67); (2) 20 mg simvastatin co-administered with niacin and laropiprant daily for 4 weeks, followed by 40 mg simvastatin co-administered with niacin and laropiprant daily for 8 weeks (N=53); (3) 40 mg simvastatin co-administered with niacin and laropiprant daily for 12 weeks (N=59), and (4) only niacin and laropiprant daily for 12 weeks (with no simvastatin administration) (N=54).

It is evident from FIG. 2 that the change in the level (an increase) of PCSK9 is associated with sustained exposure to a statin, namely, simvastatin in the present case. Thus, PCSK9 levels can be monitored to confirm whether a patient is complying with a treatment regimen including a statin.

### Example 3

### Baseline PCSK9 Levels are Indicative of Therapeutic Response to Treatment with a Statin

Samples and data were collected as described in Example 1.

It was found that subjects with a higher level of PCSK9 at Week 0 were associated with clinically poorer response to therapy with a statin for primary hypercholesterolemia or mixed hyperlipidemia. The majority of subjects exhibited clinically beneficial changes in blood parameters between Week 0 and Week 12, increases in levels of high density lipoprotein cholesterol (HDL-C) and apolipoprotein A1, and such as decreases in levels of low density lipoprotein cholesterol (LDL-C) and apolipoprotein B. However, it was found that subjects with high levels of PCSK9 at Week 0 exhibited relatively more modest increases in HDL-C and apolipoprotein A1 and relatively more modest decreases in LDL-C and apolipoprotein B.

A separate regression model for each of the parameters of HDL-C, LDL-C, apolipoprotein A1 and apolipoprotein B was evaluated. In each regression model, percent change in PCSK9 from Week 0 to Week 12 was on the left side of the equation, and on the right side of the equation each such regression model included the factors of PCSK9 at Week 0, treatment group, gender, age (as a continuous variable), geographical region (United States or non-United States), and Week 0 value of the parameter (i.e. of HDL-C, LDL-C, apolipoprotein A1 and apolipoprotein B; whichever was being evaluated). For this exercise, the following two treatment groups were combined: (i) 20 mg simvastatin administered daily for 4 weeks, followed by 40 mg simvastatin administered daily for 8 weeks (N=67), and (ii) 40 mg simvastatin administered daily for 12 weeks (N=71). Table 1 summarizes the results of these regression analyses.

TABLE 1
Regression coefficient (slope) of Week 0 PCSK9
level for prediction of percent change from Week
0 to Week 12 in the indicated parameter.

| | Slope | 95% Confidence |
|---|---|---|
| Parameter | (Standard Error) | Interval |
| HDL-C | −2.59 (1.00) | −4.56 to −0.63 |
| apolipoprotein B | +1.93 (0.95) | +0.06 to +3.80 |
| LDL-C | +1.38 (1.05) | −0.69 to +3.45 |
| apolipoprotein A1 | −1.49 (1.10) | −3.64 to +0.67 |

From the table, it is observed that for every 1 nanomolar (nM) increase in PCSK9 level at Week 0, the increase from Week 0 to Week 12 in HDL-C is attenuated by 2.59%, after controlling for the other factors included in the regression model. Because a larger increase in HDL-C is generally clinically desirable for treatment of hypercholesterolemia and hyperlipidemia, these results indicate that higher baseline values or levels of PCSK9 are associated with poorer response to the pharmacological statin therapy. Such information can inform a physician or health care professional in therapeutic decisions of drug dosage and/or frequency, drug selection, and the like. Similarly, from the table, it is observed that for every 1 nM increase in PCSK9 level at Week 0, the decrease from Week 0 to Week 12 in LDL-C is attenuated by 1.38%, after controlling for the other factors included in the regression model. Because a larger decrease in LDL-C is generally desirable clinically for hypercholesterolemia and hyperlipidemia, these results also indicate that higher baseline values or levels of PCSK9 are associated with poorer response to the pharmacological statin therapy.

### Example 4

### Identification of PCSK9 as a Putative Cardiovascular Risk Biomarker

The purpose of the present study was to improve the detection of individuals at highest risk by focusing on those who develop myocardial infarction (MI) within four years after risk assessment. Risk factors for near-term cardiovascular events like MI dominated by thrombosis superimposed on inflamed and ruptured atherosclerotic plaques could differ from risk factors for longer-term events dominated by slow development of atherosclerosis. For this purpose, a large community-based, prospective, nested case-control study was used, namely the Copenhagen City Heart Study combined with the Copenhagen General Population Study drawing upon 45,735 men and women.

Participants were from the 2001-2003 examination of the Copenhagen City Heart Study and from the 2003-2007 examination of the Copenhagen General Population Study. The Copenhagen City Heart Study is a prospective cardiovascular population study of the Danish general population initiated in 1976 comprising white men and women of Danish descent attending one or several examinations. (Nordestgaard et al., A. Nonfasting triglycerides and risk of myocardial infarction, ischemic heart disease, and death in men and women, JAMA, 298:299-308 (2007)) During the 2001-2003 examination, blood samples were collected from 5907 individuals (50% participation rate). The Copenhagen General Population Study (CGPS) is a prospective study of the Danish general population initiated in 2003 and still recruiting. (Nordestgaard et al., Nonfasting triglycerides and risk of myocardial infarction, ischemic heart disease, and death in men and women, JAMA, 298:299-308 (2007); Frikke-Schmidt et al., Association of loss-of-function mutations in the ABCA1 gene with high-density lipoprotein cholesterol levels and risk of ischemic heart disease, JAMA, 299:2524-32 (2008)), the aim is to total 100,000 participants ascertained exactly as in The Copenhagen City Heart Study. Between 2003 and 2007, 39,828 individuals from the Copenhagen General Population Study returned blood samples (45% participation rate).

Within four years of blood draw in the combined studies, 252 participants with incident nonfatal or fatal MI were identified. Controls were matched to cases from the same study, randomly selected in a 2:1 ratio from participants with a blood sample and without a history of MI (but they could previously have had other cardiovascular diseases or revascularization procedures), and matched for age (within 1 year), gender, year of examination and of blood draw (within 1 year), and HMG-CoA reductase inhibitor use (yes or no).

Information on diagnoses of MI (World Health Organization, International Classification of Diseases, 8th edition: codes 410; 10th edition: codes 121-122) was collected and verified by reviewing all hospital admissions and diagnoses entered in the national Danish Patient Registry; medical records from hospitals and general practitioners were used to verify MI diagnoses that required the presence of at least two of the following criteria: characteristic chest pain, elevated cardiac enzymes, and electrocardiographic changes indicative of MI. Five cases were only able to be matched to one control instead of two. A total of 252 cases and 499 controls were thus available for analysis.

These studies were approved by Herlev Hospital and by Danish ethical committees. Participants gave written informed consent.

For the present experiment, not all 252 blood plasma samples from participants with incident nonfatal or fatal MI (‘case’ subjects) and 499 blood plasma samples from control subjects were analyzed. Instead, multiple plasma specimens were chosen from the 252 case subjects and these specimens were pooled together to create pooled specimens. Twenty-five (25) such pooled specimens were created. Patients within the same pool will be all females or all males, developing MI with similar time-windows, with similar smoking status, and free of diabetes. Patients and matching controls will have similar smoking status (never or past smoking status and current smokers), and similar survival (0-1 year, ≧1 year).

The first step was to deplete abundant proteins from plasma in order to facilitate a good dynamic range of plasma protein measurements. A dual affinity depletion strategy was implemented. In the first stage, 14 highly abundant proteins (serum albumin, IgG, fibrinogen, transferrin, IgA, IgM, haptoglobin, alpha-2-macroglobulin, alpha-1-acid glycoprotein, alpha-1-antitrypsin, Apo A-I, Apo A-II, complement C3, and Apo B-100) were depleted by an IgY antibody column. The flow-through was further depleted by a SuperMix column that retains 50-60 moderately abundant proteins in plasma and allows significant enrichment of low abundant plasma proteins in the SuperMix flow-through fraction (Qian et al., Enhanced detection of low abundance human plasma proteins using a tandem IgY12-SuperMix immunoaffinity separation strategy, Mol Cell Proteomics, 7:1963-1973 (2008)). Following abundant protein depletion, the remaining proteins were extracted away from non-proteinaceous components by reversed-phase chromatography. The proteins then were reduced, alkylated (cysteine residues) and digested with trypsin. The resulting peptide pool was labeled with the amine specific iTRAQ reagents (Applied BioSystems, Inc.). Eight samples labeled with eight different isotope-coded versions of the iTRAQ reagent were combined into eight-plex iTRAQ mixes and were analyzed as a single sample using mass spectrometry.

Each iTRAQ mix was made up of six primary samples labeled with the iTRAQ reagents that yield marker fragment ions at m/z 114, 115, 116, 118, 119 and 121, and two QC or reference samples labeled with the iTRAQ reagents that yield the m/z 113 and 117 marker ions. Each iTRAQ mix was analyzed by two-dimensional LC-MS/MS. iTRAQ mixes were pre-fractionated by strong cation exchange into six fractions that were further separated by HPLC.

HPLC-MS generally employs online ESI MS/MS strategies. Here, however, an off-line LC-MALDI MS/MS platform was used that results in better concordance of observed protein sets across the primary samples without the need of injecting the same sample multiple times (Liu et al., A model for random sampling and estimation of relative protein abundance in shotgun proteomics, Analytical Chemistry, 76:4193-201 (2004); Sadygov et al., Statistical models for protein validation using tandem mass spectral data and protein amino acid sequence databases, Analytical Chemistry, 76:1664-71 (2004)). Following first pass data collection across all iTRAQ mixes (because the peptide fractions were retained on the MALDI target plates), the samples were analyzed a second time using a targeted MS/MS acquisition pattern derived from knowledge gained during the first acquisition. In this manner, maximum observation frequency for all of the identified proteins is accomplished (ideally, every protein should be measured in every iTRAQ mix).

Relative quantification of peptides was carried out by determining relative ion intensities between the sample specific (m/z 114, 115, 116, 118, 119, 121) and reference sample specific reporter fragments (m/z 113, 117). Using two replicates of the reference sample in each mix affords more precise measurements by averaging intensity ratios relative to the 113 and 117 reporter peaks.

Identification of peptides from the MS/MS spectra was achieved using the Mascot database searching tool (Perkins et al., Probability-based protein identification by searching sequence databases using mass spectrometry data, Electrophoresis, 20:3551-67 (1999)). After the first pass of data collection was finished, peptides were assigned to a minimum non-redundant protein set. After the completion of the second pass of MS/MS acquisitions, data were normalized using a procedure described by Vandersompele et al. (Vandesompele J et al., Accurate normalization of real-time quantitative RT-PCR data by geometric averaging of multiple internal control genes, Genome Biol., 3:34 (2002)). Quantification of proteins were achieved by assigning the median ratio of unique peptides mapped to the given protein. Three peptides assigned to the protein PCSK9 were detected and measured: CAPDEELLSCSSFSR (SEQ ID NO:1), DVINEAWFPEDQR (SEQ ID NO:2), and ILHVFHGLLPGFLVK (SEQ ID NO:3). In the preceding peptide sequences, each letter corresponds to one amino acid (Hausman et al., The cell: a molecular approach, Washington, D.C: ASM Press, ISBN 0-87893-214-3 (2004)).

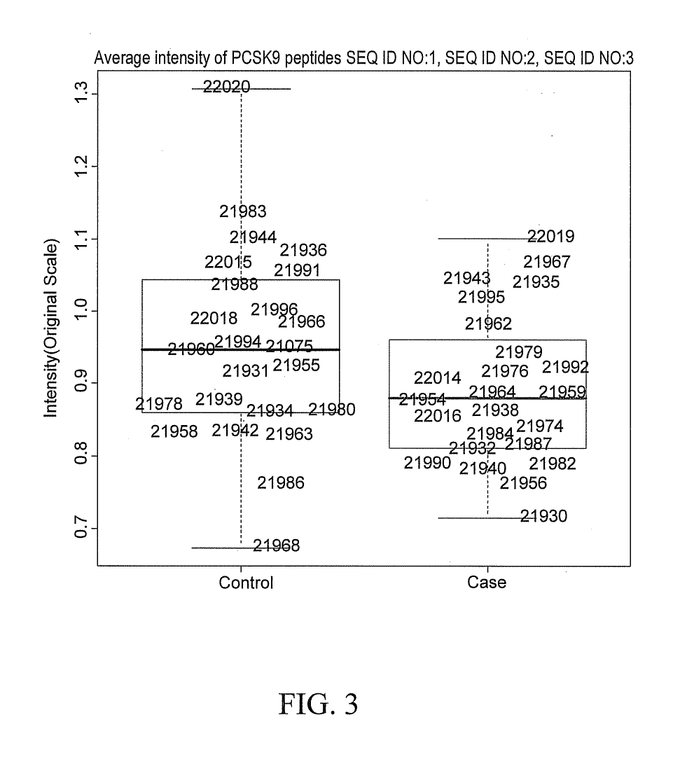

Referring to FIG. 3, a boxplot is shown of the measured plasma concentration of PCSK9, as based on the measurement of three proteolytic peptides of PCSK9 measured by mass spectrometry (namely, CAPDEELLSCSSFSR (SEQ ID NO:1), DVINEAWFPEDQR (SEQ ID NO:2), and ILHVFHGLLPGFLVK (SEQ ID NO:3)) in the 25 pooled case specimens and the 25 pooled control specimens. The level of PCSK9 was lower in abundance in the samples derived from patients who had a MI within 4 years after blood collection. The mean-fold change between cases and controls, or the ratio of the level of PCSK9 in cases to the level in controls, was measured to be 0.95, and the 95% confidence interval was 0.88 to 1.02, by a paired Student's t-test.

In sum, the measured PCSK9 levels—i.e., levels as derived from the mass spectrometric measurement of three proteolytic peptides of PCSK9—were lower in individuals who suffered a myocardial infarction within four years after sample collection. Although contrary to the general understanding of deleterious levels of total PCSK9 in the blood, these results are repeatable and evidence that PCSK9 levels are predictive of, and therefore can be used to, diagnose the risk of a patient having a cardiovascular event, such as, for example, near-term MI.

The use of headings and sections in the application is not meant to limit the present teachings; each section can apply to any aspect, embodiment, or feature of the present teachings.

Throughout the application, where compositions are described as having, including, or comprising specific components, or where processes are described as having, including or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components, or can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes,” “including,” “have,” “has,” “having,” “contain,” “contains,” or “containing” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise. As used herein, the term “about” refers to a ±10% variation from the nominal value, unless otherwise indicated or inferred.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the present teachings as if each value were specifically enumerated herein. In addition, smaller ranges between and including the upper and lower limits of a given range are contemplated and encompassed within the present teachings. The listing of exemplary values or ranges is not a disclaimer of other values or ranges between and including the upper and lower limits of a given range.

Variations, modifications, and other implementations of what is described herein will occur to those of ordinary skill in the art without departing from the spirit and the essential characteristics of the present teachings. Accordingly, the scope of the invention is to be defined not by the preceding illustrative description but instead by the following claims, and all changes that come within the meaning and range of equivalency of the claims are intended to be embraced therein.

What is claimed is:

1. A method of predicting response to treatment with a statin, the method comprising:
measuring a proprotein convertase subtilisin kexin type 9 (PCSK9) level in a sample obtained from a patient prior to the patient taking a statin or after a sufficient washout period has elapsed for the patient,
wherein a measured PCSK9 level failing within a predetermined target range is indicative that the patient is likely to respond favorably to treatment with a statin, and
wherein a measured PCSK9 level above the predetermined range is indicative that the patient is likely to have an attenuated response to treatment with a statin.

2. The method of claim 1, wherein the predetermined target range is between about 0 nM to about 7 nM.

3. The method of claim 1, wherein the measured PCSK9 level is an absolute concentration.

4. The method of claim 1, wherein the measured PCSK9 level is a normalized concentration.

5. The method of claim 1, wherein the sufficient washout period is about 6 weeks.

6-18. (canceled)

19. A computer system for predicting patient response to treatment with a statin, the computer system comprising:
an electronic memory device; and
an electronic processor in communication with the memory device wherein the memory device comprises instructions that when executed by the processor cause the processor to:
query a proprotein convertase subtilisin kexin type 9 (PCSK9) level measured in a sample obtained from a patient prior to the patient taking a statin or after a sufficient washout period has elapsed for the patient;
compare the measured PCSK9 level to a threshold range; and
generate a notification indicative of whether the measured PCSK9 level is within the target range,
wherein a measured PCSK9 level falling within a predetermined target range is indicative that the patient is likely to respond favorably to treatment with a statin, and
wherein a measured PCSK9 level above the predetermined range is indicative that the patient is likely to have an attenuated response to treatment with a statin.

20-39. (canceled)

40. A method of selecting a therapy fix a human prior to administration of the therapy, the method comprising measuring a proprotein convertase subtilisin kexin type 9 (PCSK9) blood concentration in a sample from the human, thereby to determine the presence or absence of a PCSK9 blood concentration indicative of responsiveness to an inhibitor of 3-hydroxy-3-methylglutaryl-coenzyme A reductase.

41. The method of claim 40, wherein the sample comprises blood, serum or plasma.

42. The method of claim 40, further comprising repeatedly administering the inhibitor to the patient.

43-47. (canceled)

48. The method of claim 1, wherein the statin is selected from the group consisting of atorvastatin, cerivastatin, fluvastatin, lovastatin, mevastatin, pitavastatin, pravastatin, rosuvastatin, and simvastatin.

49-52. (canceled)

53. The method of claim 40, wherein the patient has a PCSK9 blood concentration determined to be within a target range.

54. The method of claim 40, wherein the patient has a PCSK9 blood concentration determined to be above a minimum threshold.

55. The method of claim 54, wherein the minimum threshold is more than 1 nanomole per liter.

56. The method of claim 54, wherein the minimum threshold is between 1 and 1.5 nanomoles per liter.

57-59. (canceled)

60. The method of claim 54, wherein the minimum threshold is more than 3 nanomoles per liter.

61. The method of claim 40, wherein the patient has a PCSK9 blood concentration determined to be below a maximum threshold.

62. The method of claim 61, wherein the maximum threshold is below 7 nanomoles per liter.

63. The method of claim 61, wherein the maximum threshold is below 6 nanomole per liter.

64. The method of claim 61, wherein the maximum threshold is below 4 nanomoles per liter.

65. The method of claim 61, wherein the maximum threshold is between 3 and 4 nanomoles per liter.

66-68. (canceled)
